(19) United States
(12) Plant Patent Application Publication
(10) Pub. No.: US 2019/0037745 P1
(43) Pub. Date: Jan. 31, 2019

(54) Climbing rose plant named 'Kornagelio'

(50) ;

(71) Applicant: W. KORDES' SOHNE Rosenschulen GmbH & Co KG, Klein Offenseth-Sparrieshoop (DE)

(72) Inventor: Wilhelm-Alexander KORDES, Klein Offenseth-Sparrieshoop (DE)

(73) Assignee: W. KORDES' SOHNE Rosenschulen GmbH & Co KG, Klein Offenseth-Sparrieshoop (DE)

(21) Appl. No.: 15/731,759

(22) Filed: Jul. 31, 2017

(51) Int. Cl. A01H 6/74

(52) U.S. Cl. PLT/114

(57) ABSTRACT

A new and distinct variety of Climbing Rose Plant, herein referred to by its cultivar name, ‘KORnagelio’, is provided which forms in abundance on a substantially continuous basis attractive, double pink colored blossoms. The vegetation is vigorous and the growth habit is climbing. Attractive glossy, dark green foliage is formed. The resistance to disease is very good. The new variety is particularly well suited for providing distinctive ornamentation in the landscape.

Botanical/commercial classification: Latin name—Rosa hybrida. Common name—Climbing Rose Plant. Varietal Denomination—‘KORnagelio’.

### SUMMARY OF THE INVENTION

The new variety of Rosa hybrida Climbing Rose Plant of the present invention was created by during May 2005 at Klein Offenseth-Sparrieshoop, Germany by artificial pollination wherein two parents were crossed which previously had been studied in the hope that they would contribute the desired characteristics. The female parent (i.e., seed parent) of the new variety was the ‘DELeri’ variety (non-patented in the United States). The male parent (i.e., pollen parent) was the (‘KORgosumu’x‘KORvanaber’) variety (non-patented in the United States).

The parentage can be summarized as follows:

‘DELerr’(‘KORgosumu’x‘KORvanaber’)

The seeds resulting from the above pollination were sown and small plants were obtained which were physically and biologically different from each other. Selective study resulted in the identification of a single plant of the new variety.

It was found that the new Climbing Rose Plant of the present invention possesses the following combination of characteristics:
(a) abundantly and substantially continuously forms attractive, double pink colored blossoms,
(b) exhibits a climbing growth habit,
(c) forms vigorous vegetation,
(d) forms attractive ornamental glossy, dark green foliage, and
(e) exhibits very good disease resistance.

The new variety well meets the needs of the horticultural industry. It can be grown to advantage as ornamentation in parks, gardens, public areas, and in residential settings. Accordingly, the plant is particularly well suited for growing in the landscape.

The new variety can be readily distinguished from its ancestors. More specifically, the ‘DELeri’ variety (i.e., seed parent) displays smaller flower size with a lower petal count than that of the new variety. Additionally, the (‘KORgosumu’x‘KORvanaber’) variety (i.e., pollen parent) exhibits cream colored flower, whereas the new variety forms pink colored flowers with a higher petal count and has stronger vigor.

The new variety has been found to undergo asexual propagation in Klein Offenseth-Sparrieshoop, Germany by a number of routes such as budding. Asexual propagation techniques in Germany have shown that the characteristics of the new variety are homogeneous, stable, and strictly transmissible by such asexual propagation from one generation to another. Accordingly, the new variety undergoes asexual propagation in a true-to-type manner.

The new variety has been named ‘KORnagelio’.

### BRIEF DESCRIPTION OF THE PHOTOGRAPHS

The accompanying photograph shows, as nearly true as it is reasonably possible to make the same in color illustrations of this character, a typical specimen of the new variety. The illustrated rose plant of the new variety was approximately five years of age and was observed during July 2014 while budded on its own roots and growing outdoors at Sparrieshoop, Germany.

FIG. 1—illustrates a specimen of a plant displaying floral buds and flowers at varying points of opening.

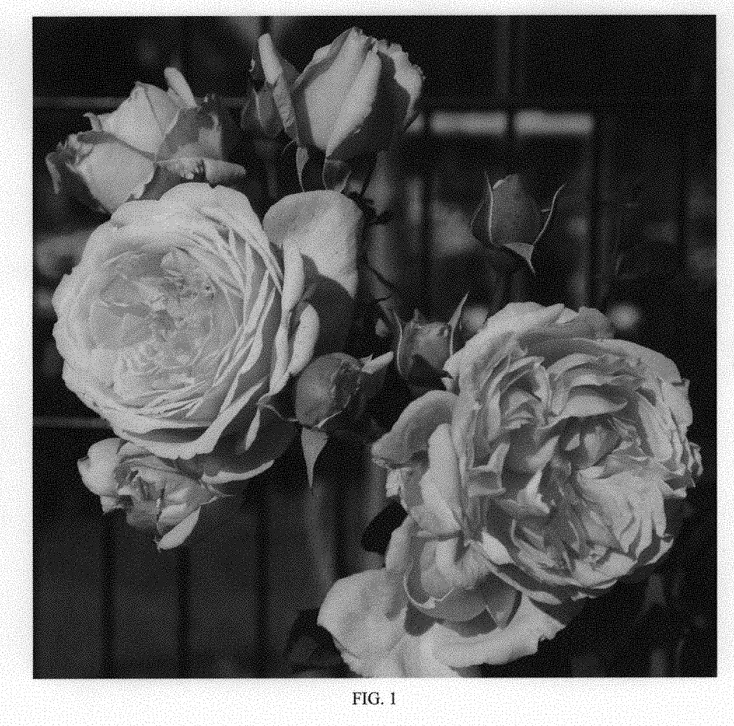

FIG. 2—illustrates a specimen of a seven-leaflet leaf obverse.

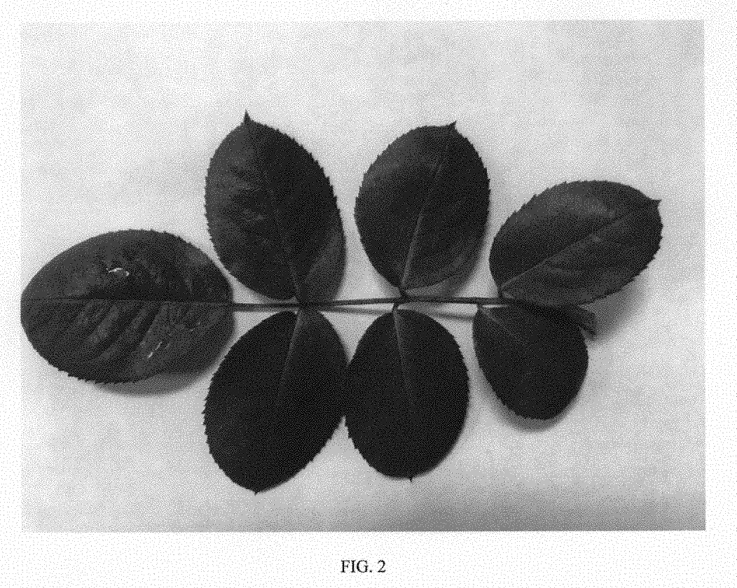

### DETAILED BOTANICAL DESCRIPTION

The chart used in the identification of colors is that of The Royal Horticultural Society (R.H.S. Colour Chart, 2015 edition). The terminology which precedes reference to the chart has been added to indicated the corresponding color in more common terms. The description is based on the observation of a three-year-old specimen of the new variety, observed during July, while growing in a three-gallon container on its own roots in Cochranville, Pa.
Class: Climbing Rose Plant.
Plant:
   Height.—approximately up to 60.0 cm from the top of the soil plane on average.
   Width.—approximately up to 40.0 cm on average.
   Habit.—climbing and upright.
Branches:
   Color.—commonly near Yellow Green Group 144A on young stems, and commonly near Yellow Green Group 146A transitioning to Greyed Green Group 197A as the wood hardens on old wood.
   Length.—main stems are approximately 60.0 cm and secondary stems are approximately 20.0 cm on average.
   Thorns.—young thorns: length is approximately 5.0 mm on average, width is approximately 5.0 mm at point of attachment on average, and color is commonly near Grey Brown Group N199C. — old thorns: length is approximately 8.0 mm on average, width is approximately 8.0 mm at point of attachment on average, and color is commonly near Grey Brown Group 199B.
Foliage:
   General appearance.—glossy, dark green.
   Young foliage.—upper surface color: commonly near Yellow Green Group 147A with thin highlights of Greyed Red Group 178A around the leaf margin. — under surface color: commonly near Yellow Green Group 147B.
   Adult foliage.—upper surface color: commonly near Yellow Green Group 147A. — under surface color: commonly near Yellow Green Group 147B.
   Petioles.—upper surface: texture is smooth, color is commonly near Yellow Green Group 146A. — under surface: texture is glandular with some small prickles, color is commonly near Yellow Green Group 146B.
   Rachis.—color: upper surface is commonly near Yellow Green Group 146A, under surface is commonly near Yellow Green Group 146B.
   Stipules.—length: approximately 20.0 mm on average. — width: approximately 6.0 mm on average. — margin: entire to erose. — color: upper surface is commonly near Yellow Green Group 145A with lighter strip of Yellow Green Group 145C towards the midline; lower surface is commonly near Yellow Green Group 145A.
   Leaf margin.—serrate.
Leaflets:
   Number.—3, 5, and 7.
   Shape.—ovate.
   Texture.—upper surface is smooth; under surface is smooth.
   Size.—terminal leaflet: length is approximately 5.5 cm on average and width is approximately 5.0 cm on average. — lower leaflets: length is approximately 5.0 cm on average and width is approximately 3.5 cm on average. — 5-Leaflet leaf: length is approximately 13.0 cm on average and width is approximately 10.0 cm on average.
Inflorescence:
   Number of flowers.—approximately 3 blooms on average on a plant at once.
   Number of blooms per stem or in a cluster.—commonly between 1 and 3 blooms per stem on average.
   Peducle.—color: commonly between Yellow Green Group 144A and Yellow Green Group 144B. — diameter: approximately 4.0 mm on average. — length: approximately 5.5 cm on average. — surface texture: sparely covered in short, flexible thorns that measure less than 2.0 mm in length.
   Sepals.—number: commonly 5. — upper surface color and texture: covered in short pubescence and commonly near Yellow Green Group 144A. — under surface color and texture: puberulent, commonly near Yellow Green Group 144B with highlights commonly near Greyed Orange Group 176A towards middle and upwards to the apex. — size: length is approximately 1.6 cm on average and width is approximately 7.0 mm on average. — margin: entire.
   Flower.—double pink colored. — form: double, cuplike. — diameter: approximately 7.0 cm on average. — color: upper and under surface is commonly near Red Purple Group 62C.
   Fragrance.—very strong sweet fragrance.
   Petal.—number: approximately 90 on average. — drop: good. — inner petals: length is approximately 1.5 cm on average and width is approximately 1.0 cm on average. — outer petals: length is approximately 3.0 cm on average and width is approximately 2.2 cm on average. — shape: overall shape is broadly obovate; apex shape is round; and base shape is cuneate. — margin: entire.
   Petaloids.—number: approximately 5 per flower on average. — color: upper surface and under surface are commonly near Red Purple Group 62C. — size: length is approximately 1.0 cm on average and width is approximately 8.0 mm on average. — texture: smooth. — margins: variable, entire to erose. — shape: overall shape is variable, oblong and mostly curving inward; apex is round, and base is cuneate.
   Stamen.—number: approximately 90 on average. — anthers: number is about 90 and color is commonly near Yellow Orange Group 16B. — filaments: length is approximately 5.0 mm on average and color is commonly near Yellow Orange Group 16D.
   Pistils.—arrangement: separate and free. — number: approximately 100 on average. — style: color is commonly near Greyed Yellow Group 160C at the base transitioning to near Greyed Red Group 180C at the apex and length is approximately 7.0 mm on average. — stigma: color is commonly near Greyed Yellow Group 161B and diameter is commonly less than 1.0 mm on average.
   Receptacle.—achenes stand on the bottom and wall; diameter is approximately 1.0 cm on average, shape is round, color is commonly near Yellow Green Group 144A, and surface texture is smooth.
   Pollen.—amount is moderate and color is commonly near Yellow Orange Group 22A.
   Hips/seed.—none observed.
Development:
   Vegetation.—dark green, vigorous and glossy.
   Blooming.—abundant and substantially continuous from spring through frost.
   Tolerance to diseases.—very good.

Plants of the ‘KORnagelio’ variety have not been observed under all possible environmental conditions to date. Accordingly, it is possible that the phenotypic expression may vary somewhat with changes in light intensity and duration, cultural practices, and other environmental conditions.

I claim:

1. A new and distinct Climbing Rose Plant characterized by the following combination of characteristics:
(a) abundantly and substantially continuously forms attractive, double pink colored blossoms,
(b) exhibits a climbing growth habit,
(c) forms vigorous vegetation,
(d) forms attractive ornamental glossy, dark green foliage, and
(e) exhibits very good disease resistance;
substantially as herein shown and described.
